(19) United States
(12) Patent Application Publication
(10) Pub. No.: US 2014/0194961 A1
(43) Pub. Date: Jul. 10, 2014

(54) Garment and method for use for modifying body temperature using blood in the circulatory system as a heat transfer fluid

(71) Applicant: Dwaine Drew Evans, JR., Flatwoods, KY (US)

(72) Inventor: Dwaine Drew Evans, JR., Flatwoods, KY (US)

(21) Appl. No.: 13/736,232

(22) Filed: Jan. 8, 2013

(51) Int. Cl. A61F 7/10; A61F 7/08

(52) U.S. Cl. CPC A61F 7/106; A61F 7/08
USPC 607/112

(57) ABSTRACT

Clothing and an associated method of use for modifying body temperature by transdermally modifying blood temperature comprising having a thermal device retained within at least one garment pocket substantially overlying at least one major blood vessel so as to increase the temperature gradient of said blood across said at least one major blood vessel so as to use said blood to act as a heat transfer fluid, said garment being fitted so as to press said pocket and said thermal device closely to the skin of the wearer and said pocket being arranged to be substantially aligned along the length of said major circulatory vessel while minimizing overlap across tissue surrounding said major blood vessel.

### CROSS REFERENCE TO RELATED APPLICATIONS

The present application relates to U.S. Provisional Patent Application 61/676,331 filed Jul. 27, 2012. The Applicant hereby incorporates U.S. Provisional Patent Application 61/676,331 as if fully set forth herein.

### TECHNICAL FIELD

The device of the present application relates generally to active wear clothing. More specifically, the device of the present application relates to active wear clothing capable of modifying the thermal environment of the wearer.

### BACKGROUND

The cardiovascular system is the primary means by which the body distributes biological heat, i.e. bioheat. Heat production and heat loss are controlled in two ways. First, the transfer of heat to the body skin surface from the central core establishes an internal thermal gradient. Second, heat is dissipated from the skin surface to the surrounding environment. This balance is critical to normal thermoregulatory function. Body temperature regulation is controlled almost exclusively by intricate nervous system feedback mechanisms located in the hypothalamus. Heat-sensitive neurons located in the pre-optic area of the hypothalamus are the body's most influential temperature receptors. These receptors respond to rising temperature by increasing their impulse output and to falling temperature by decreasing their output.

Additional temperature receptors found in the skin consist of both warmth and cold receptors. There are 4 to 10 times as many cold as warmth receptors. These receptors convey nerve impulses to the hypothalamus, where the information is used to regulate body temperature. Receptors in the spinal cord itself, the abdomen, and other internal body structures also transmit signals, primarily cold signals, to the CNS to help in temperature control. Peripheral thermoreceptors dispatch signals to the posterior hypothalamus, where they are integrated to control heat loss and heat production. This “hypothalamic thermostat” is the primary temperature control mechanism in the body.

The blood serves as a heat transfer fluid regulating core body temperature by increasing blood flow to the extremities for cooling and by diverting blood from the extremities to conserve heat and to better maintain core body temperature in frigid conditions. Core body temperature depends on many variables such as the temperatures to which the extremities are exposed and the duration of that exposure, temperature difference between extremities and core, volume of blood which deviates from core temperature, and the rate of blood perfusion. Human blood has a thermal conductivity similar to that of water, but is somewhat dependent upon hematocrit values.

An increase in the body core temperature (hyperthermia) from approximately 36.5 to 39 degrees Celsius is known to cause a doubling of the cardiac output. In connection with vasoconstriction in the splanchnic circulation and in skeletal muscle this results in large increases of skin blood flow. The underlying vasodilatation is evoked by reflex regulation of the efferent sympathetic system. While there is a reduction of alpha-adrenergic vasoconstriction, there is also evidence for active sympathetic cholinergic and nitric oxide-dependent vasodilatation. In the presence of risk factors, e.g. age and diabetes, the circulatory adaptation to heat stress may be compromised.

Overexposure to cold can result in abnormal CNS responses and varies with the degree of hypothermia. Mild to moderate hypothermia is known to produce confusion and behavioral changes, even paradoxical shedding of clothes which exacerbates the problem of overexposure. Adverse cardiological effects are also prevalent in hypothermia cases. During the phase of shivering thermogenesis, there is a decrease in cardiac conductivity and automaticity and an increase in the refractory period.

### SUMMARY

This application relates to active wear which incorporates a temperature modification device, e.g. chemical hot pack or cold pack, positioned over and oriented along a major circulatory system artery or vein so as to optimize the overlap of the temperature modification device with the major artery or vein. Optimizing the overlap of the temperature modification device with a major artery or vein permits the wearer of the garment to use the blood flowing through the affected vessel as a heat transfer fluid to either increase or decrease core body temperature and appendage temperature more effectively, resulting in improved tolerance of heat and cold. Incremental changes in the temperature of blood flowing to the extremities by the use of supplemental heat or cold is believed by the applicant to improve comfort and performance. The residence time of blood flowing within a vessel is a critical dependent variable, therefore extending the area affected by the heating or cooling properties of the clothing along the length of a vessel can improve the efficiency of the thermal modification of the blood while minimizing the impact on surrounding tissue.

### BRIEF DESCRIPTION OF THE DRAWINGS

FIG. 1 depicts a shirt possessing pockets oriented to overlap major blood vessels.

FIG. 2 depicts an embodiment of a shirt having externally accessible pockets overlapping some major blood vessels arranged beneath the external surface of the shirt and having thermal packs inserted therein.

FIG. 3 depicts an embodiment of a shirt having externally accessible elongated pockets overlapping some major blood vessels arranged beneath the external surface of the shirt and having elongated thermal packs inserted therein.

FIG. 4 depicts an embodiment of a shirt having removably affixed pockets overlapping some major blood vessels arranged beneath the external surface of the shirt and having elongated thermal packs inserted therein.

FIG. 5 depicts a pair of shorts having pockets oriented to overlap major veins in the legs.

FIG. 6 depicts a shirt having internally accessible envelope pockets.

### DETAILED DESCRIPTION

The garment of the present application can be formed as a shirt, body suit, shorts, pants, or similar garments. The garment includes at least one means to retain a thermal device 10, e.g. a pocket 10, which is preferably a chemical pack 50 which can be actuated to generate heat through an exothermic reaction or absorb heat through an endothermic reaction. Common thermal devices 50 include, but are not limited to, plastic packs having a plurality of compartments separated by an internal seal to segregate the active components wherein the seal can be ruptured through the application of sufficient pressure. Pressure applied to a pack compartment by squeezing the compartment with the hand or otherwise compressing the compartment causes the internal seal between the compartments to rupture and the components to mix, thus initiating the thermal event from the chemical reaction.

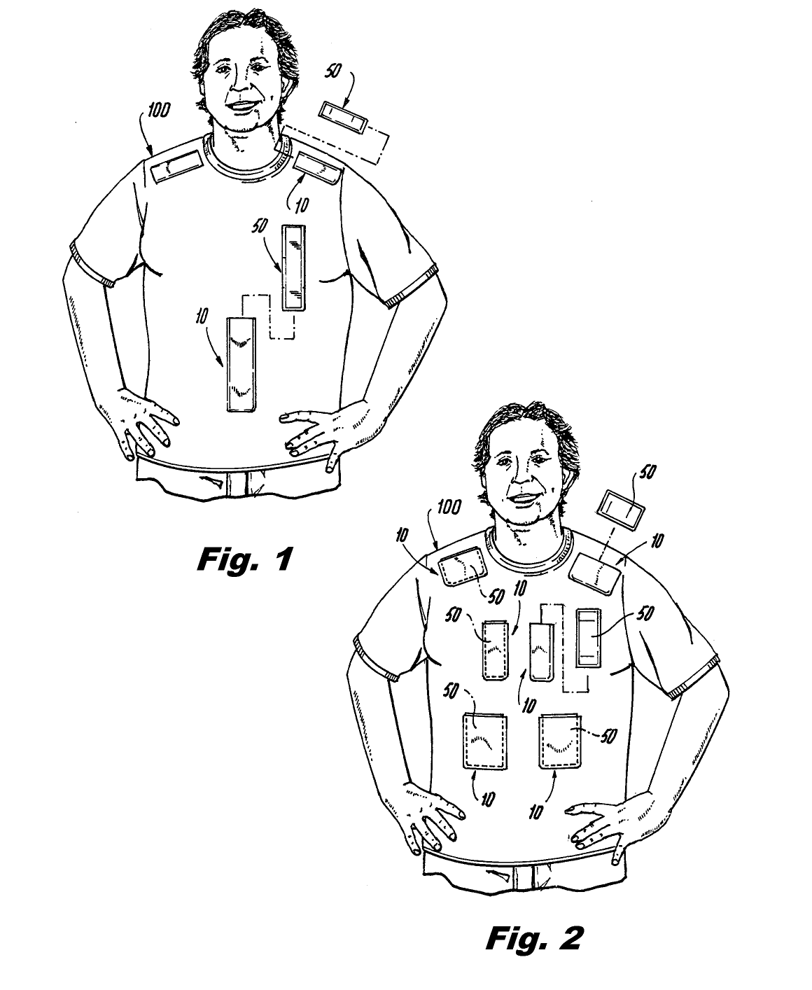

As depicted in FIG. 1, the means to retain a thermal device 50 is preferably a garment pocket 10. The pocket 10 can be incorporated into the garment 100 so as to permit it to be accessed from the outside of the garment 100 when worn, or alternatively it may be configured to be accessed from within the garment 100 when worn. In a still further embodiment, the pockets 10 are removably attached within the garment 100. Common means of removable attachment 5 for the pocket 10 include hoop and loop fabric, snaps, zippers, buttons, and similar means of removable attachment known to those skilled in the art. In another embodiment, as depicted in FIG. 4, the pockets 10 are removably affixed within said garment 100, but positionable to permit the wearer some flexibility in the placement and orientation of the pocket 10 to accommodate a wide variety of physiques. Alternatively the pockets 10 may be “envelope pockets” with one end of the pocket opening 11 tucked under an opposing pocket opening.

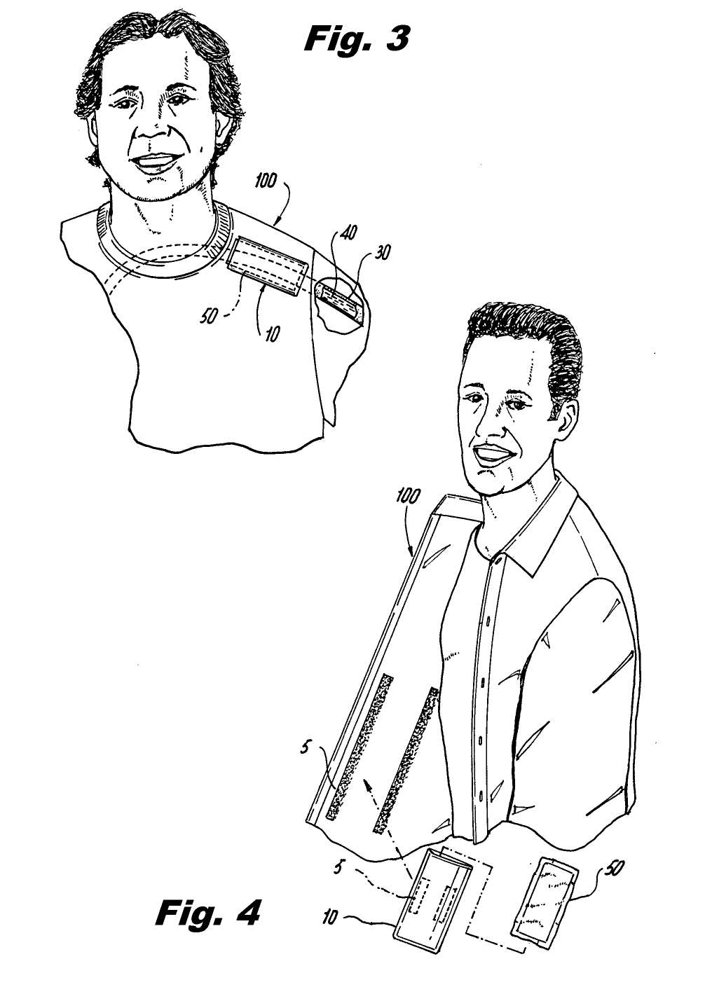

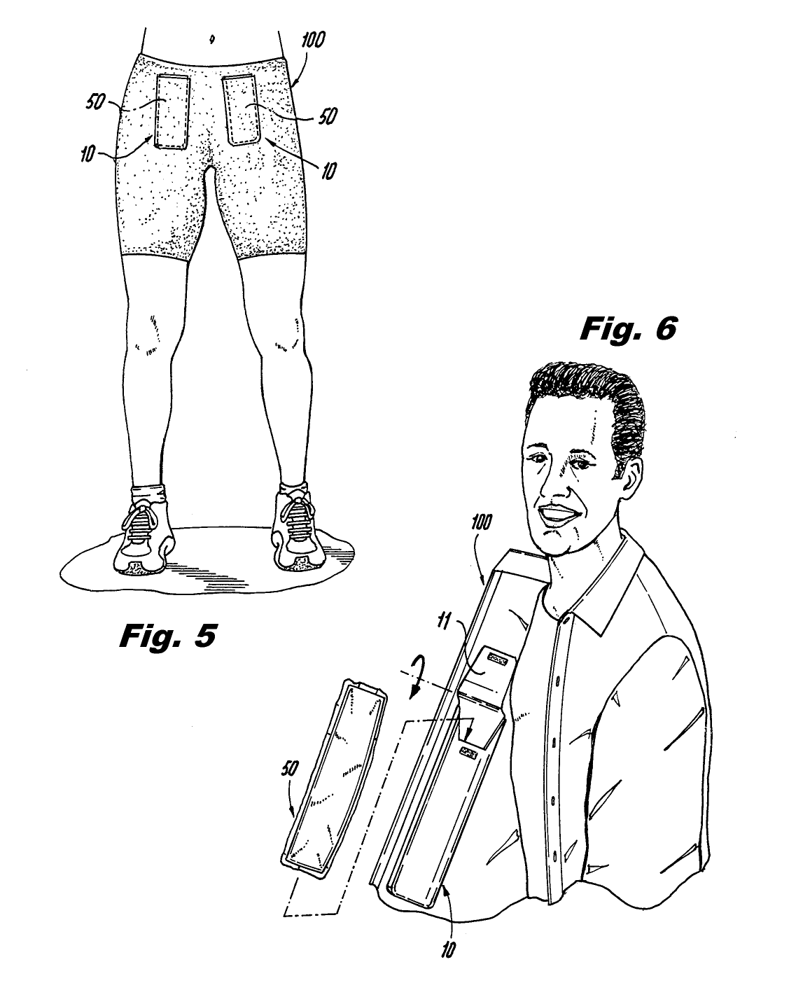

As depicted in FIG. 3, pockets 10 are ideally placed on the garment 100 to significantly overlap major blood vessels 30. Major human arteries include the hypogastric, femorals, popliteals, brachials, subclavians, carotids, abdominal aorta, brachiocephalic, thoracic aorta, axillaries, popliteals, tibials, radials, superior vena cava, and common iliac arteries. Major human veins include the superior vena cava, interior jugulars, exterior jugulars, subclavians, axillaries, brachiocephalic cephalic, basilic, cubitals, antebrachials, hepatic, renal, inferior vena cava, common iliac, external iliac, internal iliac, deep femoral, femoral, great saphenous, small saphenous, popliteal, anterior tibial, and posterior tibial. The overlap of a pocket 10 along a major blood vessel 30 permits the actuated thermal device 50 contained therein to modify the temperature of the blood 40 as it flows through the blood vessel 30 beneath the thermal device 50. The temperature modified blood 40 acts as a heat transfer fluid which circulates to the extremities, e.g. hands, feet, etc. . . . , and the body core via the circulatory system. In an embodiment, the pockets 10 will use elongated, long lasting thermal packs within a garment pocket 10 aligned along a major blood vessel 30. The elongated thermal packs 50 increase the residence time of the blood 40 in the transdermal heating zone beneath the thermal pack. The elongation of the thermal pack 50 also serves to focus the heating or cooling event in a narrow zone around the blood vessel 30 thus preventing the overuse of expensive reagents by avoiding areas where no heat distribution is likely to take place and minimizing the exposure of skin to large areas of heat or cold which would potentially interfere with and compete against the preferential regulation of body heat by the circulatory system. The modification of the temperature of the blood 30 as it flows through the circulatory system allows its use as a heat transfer fluid to modify the temperature profile of the circulated blood 30 by increasing the temperature gradient, i.e. ΔT, of the blood 30 across a blood vessel 40 before and after passing beneath a thermal pack.

The garment 100 of the present application permits the user to more effectively regulate body temperature by wearing the garment 100 and actuating thermal packs 50 to heat or alternatively cool the blood as needed. In cold climates, outdoor workers and enthusiasts would use actuate heat packs 50 inserted into the pockets 10 to add some heat to the blood to avoid the rapid cooling taking place at the extremities which would permit extended exposure while delaying the onset of hypothermia and frostbite thus delaying symptoms such as the loss of dexterity and poor decision making. Conversely, cooling packs 50 could be actuated in the pockets 10 for use in hot weather conditions to help regulate core body temperature.

1. A garment comprising a fabric and at least one means to retain a thermal device transdermally overlapping at least one major blood vessel.

2. The garment of claim 1, wherein said thermal device is actuated by a chemical reaction.

3. The garment of claim 2, wherein said chemical reaction is endothermic.

4. The garment of claim 2, wherein said chemical reaction is exothermic.

5. The garment of claim 1, wherein said means to retain said thermal device is a pocket.

6. The garment of claim 5, wherein said pocket has a pocket opening outside of said garment.

7. The garment of claim 5, wherein said pocket has a pocket opening inside of said garment.

8. The garment of claim 7, wherein said pocket is removably attached to said garment.

9. The garment of claim 5, wherein said pocket is aligned substantially along the length of said at least one major circulatory vessel so as to orient a temperature modifying device along the length of said major circulatory vessel.

10. The garment of claim 9, wherein said pocket is elongated.

11. The method of modifying body temperature by transdermally modifying blood temperature comprising having a thermal device retained within at least one garment pocket substantially transdermally overlapping at least one major blood vessel so as to increase the temperature gradient of said blood across said at least one major blood vessel so as to use said blood to act as a heat transfer fluid, said garment being fitted so as to press said pocket and said thermal device closely to the skin of the wearer and said pocket being arranged to be substantially aligned along the length of said major circulatory vessel while minimizing overlap across tissue surrounding said major blood vessel.

12. The method of claim 11, wherein said garment is used with actuated heat packs to delay the onset of the detrimental effects of hypothermia for the wearer.

13. The method of claim 12, wherein said garment is used with actuated cold packets to delay the onset of detrimental effects of hyperthermia for the wearer.
